(19) United States
(12) Patent Application Publication
(10) Pub. No.: US 2026/0088158 A1
(43) Pub. Date: Mar. 26, 2026

(54) AUTOMATED CASE COSTING SYSTEM FOR SURGICAL PROCEDURES WITH STAFF TRACKING AND REAL-TIME COST ANALYSIS

(71) Applicant: Ospitek, Inc., San Diego, CA (US)

(72) Inventors: Slim Souissi, San Diego, CA (US); Heithem Souissi, Las Vegas, NV (US)

(73) Assignee: Ospitek, Inc., San Diego, CA (US)

(21) Appl. No.: 18/896,895

(22) Filed: Sep. 26, 2024

(51) Int. Cl. G16H 40/20; G06Q 40/12

(52) U.S. Cl. CPC G16H 40/20; G06Q 40/125

(57) ABSTRACT

A system and method of automated case costing for surgical procedures can include the operations of tracking a real time location of a plurality of staff within a surgery center using wearable tracking devices that monitor the real time location of staff members among the plurality of staff within the surgery center to provide tracked time information, and allocating staffing costs based on actual time spent in an operating room and in other areas of the surgery center including one or more among a waiting room, a Pre-Op area, a recovery area, and a post-recovery area using one or more processors. Such a system can use and integrate with human resources clocking and surgery scheduling software modules.

### CROSS-REFERENCE TO RELATED APPLICATION

Not applicable.

### TECHNICAL FIELD

The present disclosure is in the field of healthcare management. More particularly, the present disclosure provides systems for estimating and allocating the cost of staffing for surgical procedures in ambulatory surgery centers using real-time staff tracking and data analysis to improve the accuracy and efficiency of procedure cost estimations.

### BACKGROUND

Estimating the cost of staffing for surgical procedures in ambulatory surgery centers is a complex and labor-intensive task. Staff members, including surgeons, nurses, and support staff, may be compensated as hourly employees, W2 employees, or Per Diem employees. Currently, site administrators manually calculate the cost of each procedure by estimating the total staffing cost based on hours worked, as recorded by Human Resources (HR) clocking software, and then allocating these costs proportionally to various procedures based on their duration.

This method is not only time-consuming but also inaccurate, as it involves an even cost allocation across procedures without accounting for the exact staff involvement in each procedure. The lack of precision can lead to financial inefficiencies and negatively impact the surgery center's profitability.

### BRIEF DESCRIPTION OF THE FIGURES

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings in which:

FIG. 1 is a user interface for viewing staff information for scheduling and costs at a surgical center according to embodiments of the present disclosure;

FIG. 2 is an example of a Pre-OP patient/staffing chart for one or more surgical centers that can be part of a user interface output according to the embodiments;

FIG. 3 illustrates a block diagram integrating a staff location tracking module, a human resources module, surgery scheduling module, and cost calculation engine in accordance with the embodiments;

FIG. 4 illustrates a flow chart for a method for automated case costing for surgical procedures in accordance with the embodiments.

### SUMMARY

The present disclosure addresses the problems of automation and accuracy in estimating the cost of staffing surgical procedures by introducing an automated case costing system that leverages real-time staff tracking and advanced data analysis. The system utilizes a tracking mechanism to locate staff members within the surgery center, focusing on two primary areas: (1) the Operating Room (OR) and (2) all other locations within the surgery center, including the Waiting Room, Pre-Op, Recovery, and Post-Recovery areas.

In some embodiments, an automated case costing system for surgical procedures can include a server and one or more applications executing on the server that perform certain operations or functions. The operations can include tracking a real time location of a plurality of staff within a surgery center and allocating staffing costs based on actual time spent in an operating room and in other areas of the surgery center including one or more among a waiting room, a Pre-Op area, a recovery area, and a post-recovery area.

In some embodiments, the system further includes wearable tracking devices that monitors the real time location of staff members among the plurality of staff within the surgery center to provide tracked time information.

In some embodiments, the system further includes a data integration module that integrates with a human resources clocking software and a surgery center's scheduling software to retrieve compensation rates, staff hours, and procedure schedules.

In some embodiments, the system includes a cost calculation engine that processes the tracked time information and applies compensation rates retrieved from the data integration module to calculate a staffing cost for each procedure, wherein the cost calculation engine allocates time spent in the operating room directly to a corresponding procedure while time outside the operating room is evenly distributed across all procedures.

In some embodiments, the system further includes a real time cost monitoring module including a cost visualization interface that that displays a real-time cost curve, shows the aggregate staffing cost over time and allows administrators to view current staffing costs and comparisons against historical targets.

In some embodiments, the real time cost monitoring module further includes a target cost comparison module that uses historical data to establish target staffing costs for specific types of surgery days or surgery procedures and enabling real time benchmarking and adjustments.

In some embodiments, the system further includes a staffing optimization module that analyzes historical patient flow and staffing data to identify trends and make recommendations for optimal staffing levels.

In some embodiments, the system further includes an alert system that provides an alert when the system detects a discrepancy between current staffing and patient needs.

In some embodiments, the alert is tailored to a specific area of the surgery center including at least one or more of the operating room, the pre-OP area, and the recovery area.

In some embodiments, the one or more applications executing on the server perform the operations of allocating time spent by staff members in the operating room to a specific procedure and further allocating time spent in other areas in an evenly distributed manner across all procedures.

In some embodiments, the system further includes a real-time cost estimation feature that aggregates staff costs and displays them as a dynamic cost curve, providing insights into staffing efficiency and enabling real-time adjustments.

In some embodiments, the one or more applications executing on the server perform the operations of using historical data to identify potential overstaffing or understaffing situations and generating alerts to guide staffing decisions.

In some embodiments, the one or more applications executing on the server perform the operations of balancing nurses between Pre-Op and PACU based on staffing shortages or excesses and automatically notifying nurses to move between the two areas.

In some embodiments, the one or more applications executing on the server perform the operations of integrating staff tracking data, human resources clocking software, and surgery scheduling software to automate a calculation and allocation of staffing costs for surgical procedures.

In some embodiments, an automated case costing system for surgical procedures can include one or more processors and memory coupled to the one or more processors having computer instructions which when executed cause the one or more processors to perform certain operations. The operations can include tracking a real time location of a plurality of staff within a surgery center using wearable tracking devices that monitor the real time location of staff members among the plurality of staff within the surgery center to provide tracked time information, allocating staffing costs based on actual time spent in an operating room and in other areas of the surgery center including one or more among a waiting room, a Pre-Op area, a recovery area, and a post-recovery area.

In some embodiments, the automated case costing system can further include a data integration module that integrates with a human resources clocking software and a surgery center's scheduling software to retrieve compensation rates, staff hours, and procedure schedules.

In some embodiments, the automated case costing system can further include a cost calculation engine that processes the tracked time information and applies compensation rates retrieved from the data integration module to calculate a staffing cost for each procedure, wherein the cost calculation engine allocates time spent in the operating room directly to a corresponding procedure while time outside the operating room is evenly distributed across all procedures.

In some embodiments, the automated case costing system further includes a real time cost monitoring module including a cost visualization interface that that displays a real-time cost curve, shows the aggregate staffing cost over time and allows administrators to view current staffing costs and comparisons against historical targets.

In some embodiments, a method of automated case costing for surgical procedures can include the steps or operations of tracking a real time location of a plurality of staff within a surgery center using wearable tracking devices that monitor the real time location of staff members among the plurality of staff within the surgery center to provide tracked time information, and allocating staffing costs based on actual time spent in an operating room and in other areas of the surgery center including one or more among a waiting room, a Pre-Op area, a recovery area, and a post-recovery area using one or more processors.

In some embodiments, the method further includes the operation of retrieving compensation rates, staff hours, and procedure schedules using a data integration module that integrates with a human resources clocking software and a surgery center's scheduling software.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. Also in these instances, well-known structures may be omitted or shown and described in reduced detail to avoid unnecessarily obscuring descriptions of the embodiments.

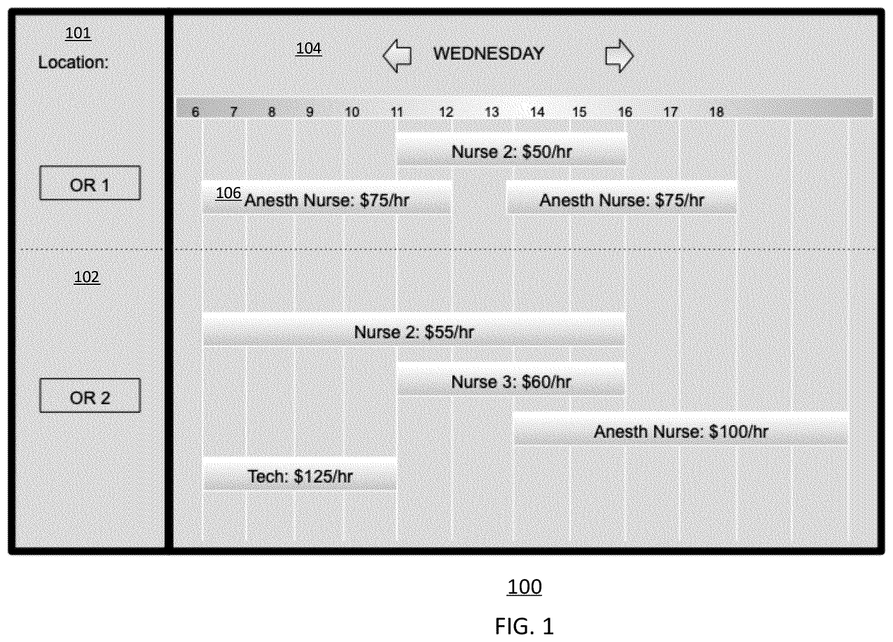

FIG. 1 illustrates a user interface 100 that enables the creation and storage of schedules for OR1 and OR2 respectively. More specifically, in the schedule 100 in FIG. 1 for OR1 or OR2 as noted in field 101 or 102 respectively, the roles in field(s) or blocks 106 for Nurse 2, Nurse 3, Anesth. Nurse, and Tech are listed, and schedules inputted as represented by blocks 106 the date shown in field 104. Also note, important information is displayed here, including the rate and position of the particular staff. The same user interface 100 can further include different color bars or other visual indicators showing the actual time that the particular staff member spent in the OR (OR1 or OR2) as providing by the location tracking system further discussed below. The data for actual time spent in the OR can be tracked with beacons, badges, wireless dongles or other location tracking devices that the staff member can wear on their person. In some embodiments, the tracking devices are wireless. In other embodiments, the tracking device can use NFC or near field communication devices that are typically found on badges. Other embodiments could use biometric sensing devices in a room that would recognize and distinguish the various individuals or staff of a team in a surgical suite. For example, cameras can use facial recognition, microphones can help perform voice recognition and identification, and scanners can do iris scanning. Obviously, some of the biometric sensing schemes may be too obtrusive for a fast paced working environment and may not provide the ideal tracking unless such sensing becomes less obtrusive. With advances in technology, some of the obtrusive schemes may no longer be obtrusive. All manner of tracking in this regard is contemplated by the embodiments herein.

In some embodiments, a user interface for scheduling staff members can come in the form of an overlay to an operating room (OR) dashboard such as the dashboard or intelligent schedule board disclosed and described in U.S. Pat. No. 11,315,678 granted on Apr. 25, 2022 to Slim Souissi et al. and assignee herein. The teaching and descriptions in U.S. Pat. No. 11,315,678 are hereby incorporated by reference in its entirety.

In some embodiments, the user interface 100 can essentially serve as an overlay to a OR dashboard that enables an operations manager or other authorized personnel to easily assign staff members intelligently to various procedures that can be occurring at one or more facilities and at one or more locations within a particular facility and to further make cost effective analysis and decisions.

Obviously, the more information that is already inputted into the system, the more intelligently the system can be able to present appropriate staff members for selection as options by the operations manager or even automatically by the system itself in certain situations. In this regard, the system can use computer or algorithmic code or a number of forms of artificial intelligence such as machine learning or deep learning to appropriately match the best (or some of the best) suited staff member candidates available to an open position and the most cost-effective rates without diminishing the overall provision of services. Again, the factors or parameters that the system can utilize for selection can include one or more among type of procedure, the number of patients, average patient wait time, the amount of experience the staff member has had with the type of procedure, doctor or surgeon preferences, staff qualifications, wage rates, staff member preferences, number of previous pairings between doctor or surgeon with staff members, prior doctor or surgeon scoring or ratings of the staff members for a particular procedure or overall, and other factors or parameters as previously described above. Note that many surgeons and OR nurses have a “chemistry” together that allows them to successfully and confidently work together. A rating system can attempt to account for such chemistry to allow for further successful pairings and outcomes. The system can use an evidence based analytical approach (like in “Moneyball” to select a baseball players for a team) to select the appropriate staff members for successful outcomes.

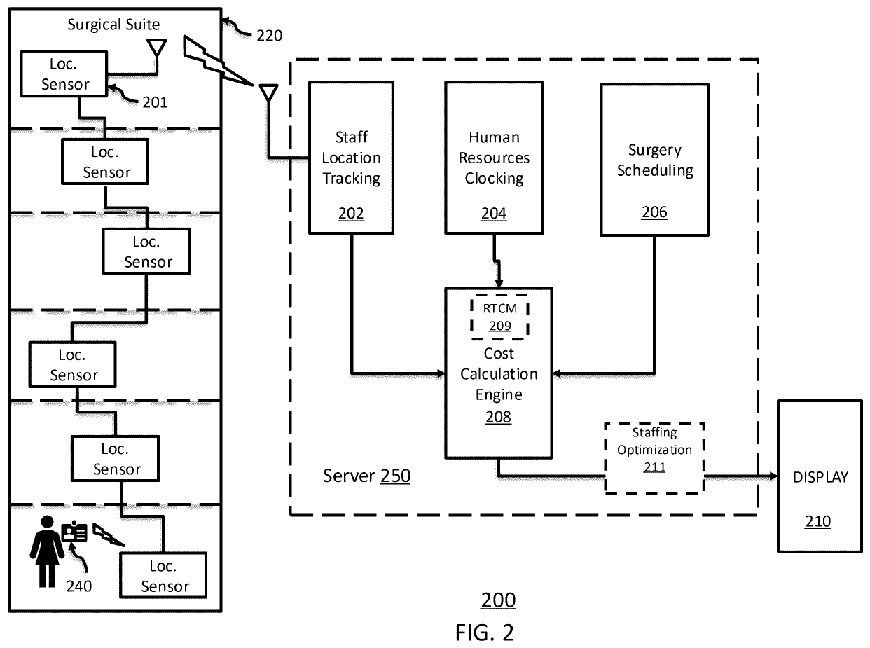

Referring to FIG. 2, a system 200 in accordance with the embodiments can include modules for automated staff tracking 202 and time allocation using a human resources or HR clocking system 204. In some embodiments, the system 200 can automatically track the location of each staff member within a surgery center 220 using one or more location sensors or detectors 201.

Time spent by staff in the Operating Room can be allocated directly to the specific procedure being performed or planned based on information that may be retrieve from a surgery scheduling module or database 306. Time spent by staff outside the Operating Room can be calculated and evenly distributed across all procedures performed during the estimation period, ensuring that all time is accounted for in the costing process.

Using a cost calculation engine 308 and data integrated from the modules 202, 204, and 206, the system 200 can provide real-time cost estimation and visualization using a display 210. In some embodiments, the system provides an instantaneous estimate of staffing costs by aggregating the cost data for each staff member present in the surgery center. A dynamic cost curve can be generated, showing fluctuations in staffing costs over time, allowing site administrators to monitor and adjust staffing levels in real-time. The curve may include a target staffing cost based on historical data for similar surgery days, enabling the administrator to assess whether the day is overstaffed or understaffed.

The system 200 can further include Staffing Optimization Alerts where the system analyzes historical staffing data and patient flow to identify potential overstaffing or understaffing situations. For example, if the Pre-Op area has only four patients but is staffed by three nurses, the system will alert the administrator of overstaffing, suggesting that only two nurses may be necessary for the current patient load. These alerts enable proactive adjustments to staffing levels, improving operational efficiency and cost control.

In some embodiments, with reference to FIG. 2 again, the system can include a staff tracking mechanism, a data integration module, and a cost calculation engine. The Staff Tracking Mechanism can include wearable devices, badges, or other tracking technologies that monitor the real-time location of staff members within the surgery center. The data integration module can integrate with HR clocking software and the surgery center's scheduling software to retrieve compensation rates, staff hours, and procedure schedules. The cost calculation engine processes the tracked time data and applies the appropriate compensation rates to calculate the staffing cost for each procedure. The engine allocates time spent in the OR directly to the corresponding procedure, while time outside the OR is evenly distributed across all procedures.

The system can then provide Real-Time Cost Monitoring that can include a cost visualization interface and targe cost comparison. The Cost Visualization Interface includes a user interface that displays a real-time cost curve, showing the aggregate staffing cost over time. This interface allows administrators to view current staffing costs and compare them against historical targets. The Target Cost Comparison would provide Historical data is used to establish target staffing costs for specific types of surgery days, enabling real-time benchmarking and adjustments.

The system can also provide for Staffing Optimization with historical data analysis and an alerting system. With respect to historical data analysis, the system analyzes historical patient flow and staffing data to identify trends and make recommendations for optimal staffing levels. When the system detects a discrepancy between current staffing and patient needs, the alerting system generates alerts to prompt adjustments. These alerts can be tailored to specific areas, such as the OR, Pre-Op, or Recovery areas.

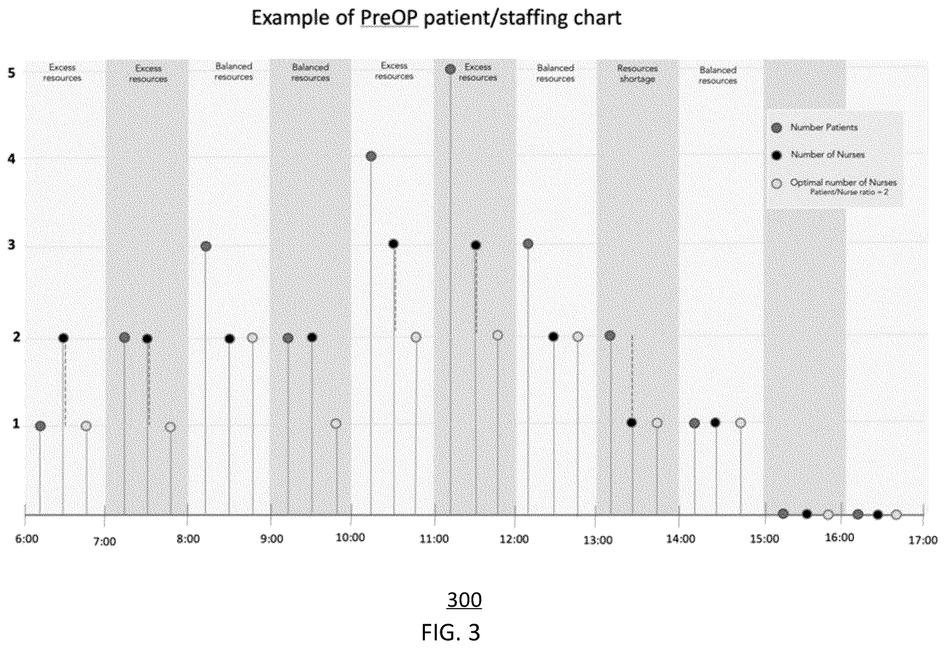

FIG. 3 depicts an example of patient/staffing chart 300. The patient to nurse staffing ratio can be set to 2, meaning for every two patients one nurse is needed. By detecting the presence of a patient or patients and nurses in the PreOp, the system can estimate shortage and excess of resources and visualize the staffing performance. This applies to all locations within the surgery center. The site administrator relies on historical data to decide about how to staff every location within the surgery center. The same chart can be used for the recovery area. Nurses who are skilled in PreOp and recovery can help balance the resource loading by moving from PACU to PreOP or vice versa based on resource excess/shortages. Nurses can be automatically notified about moving between the two areas based on live resource evaluations.

In some embodiments with reference to FIG. 2 again, an automated case costing system 200 for surgical procedures can include a server 250 and one or more applications executing on the server that perform certain operations or functions. The operations can include tracking a real time location of a plurality of staff within a surgery center and allocating staffing costs based on actual time spent in an operating room and in other areas of the surgery center including one or more among a waiting room, a Pre-Op area, a recovery area, and a post-recovery area.

In some embodiments, the system 200 further includes wearable tracking devices 240 (such as a badge, wristband, NFC card or sticker) that monitors the real time location of staff members among the plurality of staff within the surgery center 220 to provide tracked time information.

In some embodiments, the system further includes a data integration module that integrates with a human resources clocking software (204) and a surgery center's scheduling software (206) to retrieve compensation rates, staff hours, and procedure schedules.

In some embodiments, the system includes a cost calculation engine 208 that processes the tracked time information and applies compensation rates retrieved from the data integration module to calculate a staffing cost for each procedure, where the cost calculation engine allocates time spent in the operating room directly to a corresponding procedure while time outside the operating room is evenly distributed across all procedures.

In some embodiments, the system further includes a real time cost monitoring module 209 or RTCM including a cost visualization interface that that displays a real-time cost curve on a display 210, shows the aggregate staffing cost over time and allows administrators to view current staffing costs and comparisons against historical targets. In some embodiments the RTCM 209 can be part of the Cost Calculation Engine 208.

In some embodiments, the real time cost monitoring module 209 further includes a target cost comparison module that uses historical data to establish target staffing costs for specific types of surgery days or surgery procedures and enabling real time benchmarking and adjustments.

In some embodiments, the system further includes a staffing optimization module 211 that analyzes historical patient flow and staffing data to identify trends and make recommendations for optimal staffing levels.

In some embodiments, the system further includes an alert system that provides an alert when the system detects a discrepancy between current staffing and patient needs.

In some embodiments, the alert is tailored to a specific area of the surgery center including at least one or more of the operating room, the pre-OP area, and the recovery area.

In some embodiments, the one or more applications executing on the server perform the operations of allocating time spent by staff members in the operating room to a specific procedure and further allocating time spent in other areas in an evenly distributed manner across all procedures.

In some embodiments, the system further includes a real-time cost estimation feature that aggregates staff costs and displays them as a dynamic cost curve, providing insights into staffing efficiency and enabling real-time adjustments.

In some embodiments, the one or more applications executing on the server perform the operations of using historical data to identify potential overstaffing or understaffing situations and generating alerts to guide staffing decisions.

In some embodiments, the one or more applications executing on the server perform the operations of balancing nurses between Pre-Op and PACU based on staffing shortages or excesses and automatically notifying nurses to move between the two areas.

In some embodiments, the one or more applications executing on the server 250 perform the operations of integrating staff tracking data, human resources clocking software, and surgery scheduling software to automate a calculation and allocation of staffing costs for surgical procedures.

In some embodiments, an automated case costing system for surgical procedures can include one or more processors and memory coupled to the one or more processors having computer instructions which when executed cause the one or more processors to perform certain operations. In some embodiments, the one or more processors can be part of an on-premise server. In other embodiments, the storage and processing can occur in a cloud environment. In yet other embodiments, the storage and processing can by a hybrid system using both on-premise and cloud resources.

As noted previously, the operations in the automated costing system can include tracking a real time location of a plurality of staff within a surgery center 220 using wearable tracking devices that monitor the real time location of staff members among the plurality of staff within the surgery center to provide tracked time information, allocating staffing costs based on actual time spent in an operating room and in other areas of the surgery center including one or more among a waiting room, a Pre-Op area, a recovery area, and a post-recovery area.

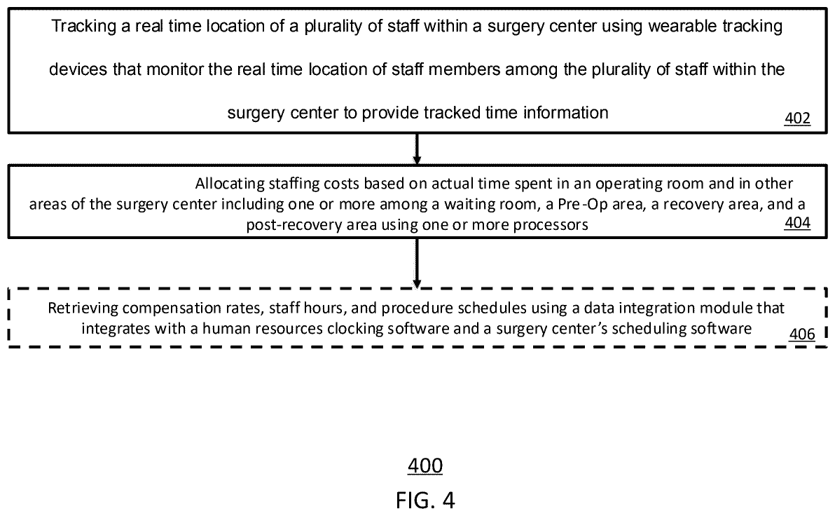

In some embodiments with further reference to FIG. 4, a method 400 of automated case costing for surgical procedures can include the steps or operations of tracking at step 402 a real time location of a plurality of staff within a surgery center using wearable tracking devices that monitor the real time location of staff members among the plurality of staff within the surgery center to provide tracked time information, and allocating at step 404 staffing costs based on actual time spent in an operating room and in other areas of the surgery center including one or more among a waiting room, a Pre-Op area, a recovery area, and a post-recovery area using one or more processors.

In some embodiments, the method 400 further includes the operation of retrieving at step 406 compensation rates, staff hours, and procedure schedules using a data integration module that integrates with a human resources clocking software and a surgery center's scheduling software.

The terms “include” and “comprise” as well as derivatives thereof, in all of their syntactic contexts, are to be construed without limitation in an open, inclusive sense, (e.g., “including, but not limited to”) . The term “or,” is inclusive, meaning and/or. The phrases “associated with” and “associated therewith,” as well as derivatives thereof, can be understood as meaning to include, be included within, interconnect with, contain, be contained within, connect to or with, couple to or with, be communicable with, cooperate with, interleave, juxtapose, be proximate to, be bound to or with, have, have a property of, or the like.

Unless the context requires otherwise, throughout the specification and claims which follow, the word “comprise” and variations thereof, such as, “comprises” and “comprising,“ are to be construed in an open, inclusive sense, e.g., ”including, but not limited to.”Reference throughout this specification to “one embodiment” or “an embodiment” or “some embodiments” and variations thereof mean that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content and context clearly dictates otherwise. It should also be noted that the conjunctive terms, “and” and “or” are generally employed in the broadest sense to include “and/or” unless the content and context clearly dictates inclusivity or exclusivity as the case may be. In addition, the composition of “and” and “or” when recited herein as “and/or” is intended to encompass an embodiment that includes all of the associated items or ideas and one or more other alternative embodiments that include fewer than all of the associated items or idea.

In the present disclosure, conjunctive lists make use of a comma, which may be known as an Oxford comma, a Harvard comma, a serial comma, or another like term. Such lists are intended to connect words, clauses or sentences such that the thing following the comma is also included in the list.

As the context may require in this disclosure, except as the context may dictate otherwise, the singular shall mean the plural and vice versa. All pronouns shall mean and include the person, entity, firm or corporation to which they relate. Also, the masculine shall mean the feminine and vice versa.

When so arranged as described herein, each computing device or processor may be transformed from a generic and unspecific computing device or processor to a combination device comprising hardware and software configured for a specific and particular purpose providing more than conventional functions and solving a particular technical problem with a particular technical solution. When so arranged as described herein, to the extent that any of the inventive concepts described herein are found by a body of competent adjudication to be subsumed in an abstract idea, the ordered combination of elements and limitations are expressly presented to provide a requisite inventive concept by transforming the abstract idea into a tangible and concrete practical application of that abstract idea.

The headings and Abstract of the Disclosure provided herein are for convenience only and do not limit or interpret the scope or meaning of the embodiments. The various embodiments described above can be combined to provide further embodiments. Aspects of the embodiments can be modified, if necessary to employ concepts of the various patents, application and publications to provide further embodiments.

What is claimed is:

1. An automated case costing system for surgical procedures, comprising:
a server; and
one or more applications executing on the server that:
tracks a real time location of a plurality of staff within a surgery center; and
allocates staffing costs based on actual time spent in an operating room and in other areas of the surgery center including one or more among a waiting room, a Pre-Op area, a recovery area, and a post-recovery area.

2. The system of claim 1, further comprising wearable tracking devices that monitors the real time location of staff members among the plurality of staff within the surgery center to provide tracked time information.

3. The system of claim 2, further comprising a data integration module that integrates with a human resources clocking software and a surgery center's scheduling software to retrieve compensation rates, staff hours, and procedure schedules.

4. The system of claim 3, further comprising a cost calculation engine that processes the tracked time information and applies compensation rates retrieved from the data integration module to calculate a staffing cost for each procedure, wherein the cost calculation engine allocates time spent in the operating room directly to a corresponding procedure while time outside the operating room is evenly distributed across all procedures.

5. The system of claim 4, wherein the system further comprises a real time cost monitoring module including a cost visualization interface that that displays a real-time cost curve, shows the aggregate staffing cost over time and allows administrators to view current staffing costs and comparisons against historical targets.

6. The system of claim 5, wherein the real time cost monitoring module further comprises a target cost comparison module that uses historical data to establish target staffing costs for specific types of surgery days or surgery procedures and enabling real time benchmarking and adjustments.

7. The system of claim 1, further comprising a staffing optimization module that analyzes historical patient flow and staffing data to identify trends and make recommendations for optimal staffing levels.

8. The system of claim 7, further comprising an alert system that provides an alert when the system detects a discrepancy between current staffing and patient needs.

9. The system of claim 8, wherein the alert is tailored to specific area of the surgery center including at least one or more of the operating room, the pre-OP area, and the recovery area.

10. The system of claim 1, wherein the one or more applications executing on the server perform the operations of allocating time spent by staff members in the operating room to a specific procedure and further allocating time spent in other areas in an evenly distributed manner across all procedures.

11. The system of claim 1, further comprising a real-time cost estimation feature that aggregates staff costs and displays them as a dynamic cost curve, providing insights into staffing efficiency and enabling real-time adjustments.

12. The system of claim 1, wherein the one or more applications executing on the server perform the operations of using historical data to identify potential overstaffing or understaffing situations and generating alerts to guide staffing decisions.

13. The system of claim 1, wherein the one or more applications executing on the server perform the operations of balancing nurses between Pre-Op and PACU based on staffing shortages or excesses and automatically notifying nurses to move between the two areas.

14. The system of claim 1, wherein the one or more applications executing on the server perform the operations of integrating staff tracking data, human resources clocking software, and surgery scheduling software to automate a calculation and allocation of staffing costs for surgical procedures.

15. An automated case costing system for surgical procedures, comprising:
one or more processors; and
memory coupled to the one or more processors having computer instructions which when executed cause the one or more processor to perform the operations comprising:
tracking a real time location of a plurality of staff within a surgery center using wearable tracking devices that monitor the real time location of staff members among the plurality of staff within the surgery center to provide tracked time information;
allocating staffing costs based on actual time spent in an operating room and in other areas of the surgery center including one or more among a waiting room, a Pre-Op area, a recovery area, and a post-recovery area.

16. The system of claim 15, further comprising a data integration module that integrates with a human resources clocking software and a surgery center's scheduling software to retrieve compensation rates, staff hours, and procedure schedules.

17. The system of claim 16, further comprising a cost calculation engine that processes the tracked time information and applies compensation rates retrieved from the data integration module to calculate a staffing cost for each procedure, wherein the cost calculation engine allocates time spent in the operating room directly to a corresponding procedure while time outside the operating room is evenly distributed across all procedures.

18. The system of claim 17, wherein the system further comprises a real time cost monitoring module including a cost visualization interface that that displays a real-time cost curve, shows the aggregate staffing cost over time and allows administrators to view current staffing costs and comparisons against historical targets.

19. A method of automated case costing for surgical procedures, comprising:
tracking a real time location of a plurality of staff within a surgery center using wearable tracking devices that monitor the real time location of staff members among the plurality of staff within the surgery center to provide tracked time information;
allocating staffing costs based on actual time spent in an operating room and in other areas of the surgery center including one or more among a waiting room, a Pre-Op area, a recovery area, and a post-recovery area using one or more processors.

20. The method of claim 19, further comprising retrieving compensation rates, staff hours, and procedure schedules using a data integration module that integrates with a human resources clocking software and a surgery center's scheduling software.
